(19) United States
(12) Patent Application Publication
(10) Pub. No.: US 2016/0220736 A1
(43) Pub. Date: Aug. 4, 2016

(54) ENZYME TRIGGERED RELEASE OF BIOACTIVE AGENTS BY LIVE CELLS

(71) Applicants: Danmarks Tekniske Universitet, Lyngby (DK); Kobenhavns Universitet, Copenhagen K (DK)

(72) Inventors: Thomas Eiland Nielsen, Charlottenlund (DK); Vitaly V. Komnatnyy, Lyngby (DK); Michael Givskov, Humlebæk (DK); Tim Tolker-Nielsen, Kokkedal (DK)

(21) Appl. No.: 14/916,452

(22) PCT Filed: Sep. 3, 2014
(86) PCT No.: PCT/DK2014/050271

§ 371 Date: Mar. 3, 2016

(30) Foreign Application Priority Data
Sep. 4, 2013 (EP) 13182983.0

(51) Int. Cl. A61L 31/10; A01N 43/60; A01N 37/02; A61L 31/16

(52) U.S. Cl. CPC A61L 31/10; A61L 31/16; A01N 43/60; A01N 37/02; A61L 2300/21; A61L 2300/404; A61L 2300/606; A61L 2420/02

(57) ABSTRACT

The present invention relates to modified polymer surfaces capable of releasing bioactive agents and a method for preventing cell growth on a surface. Thus, one aspect of the invention relates to a medical device comprising a device substrate having a surface, a polymer coating attached to said surface, and a bioactive agent covalently attached to said polymer coating, and wherein said bioactive agent is covalently attached to said polymer coating via at least one ester or carboxylic acid anhydride moiety sensitive to cleavage by an enzyme. Another aspect of the present invention relates to a method of inhibiting or preventing cell growth on a surface comprising modifying said surface with a polymer coating comprising a bioactive agent covalently attached to said polymer coating.

### TECHNICAL FIELD OF THE INVENTION

The present invention relates to modified polymer surfaces capable of releasing bioactive agents. In particular, the present invention relates to medical devices comprising a polymer surface where bioactive agents are covalently attached via specific ester bonds which may be cleaved by enzymes. Also, a method of preventing bacterial colonies or formations such as biofilms on surfaces in general is described.

### BACKGROUND OF THE INVENTION

Medical devices routinely employed in healthcare practise are often susceptible to microbial contamination or other forms of undesirable cell growth. Pathogens may attach themselves to device surfaces of catheters or implants and form microbial aggregates such as for example biofilms, which may be the direct cause of device failure. Treatment of device-related infections is often difficult as pathogens exhibit a high degree of antibiotic resistance e.g. in the biofilm form. For the most severe cases, surgery and replacement of the indwelling device is the only option to clear the infection. A press release from the Council Recommendation on patient safety (Council of the European Union, June 2009), stated that health care associated infections on average occur in one of twenty hospitalized patients, that is to say 4 million patients a year in the EU, and that 37.000 deaths are caused every year as a result of such infections.

Other surfaces such as the inner surfaces of tubes and pipelines for transporting liquid compositions or the inner surfaces of tanks or bioreactors in the food or biomedical industry may also be susceptible to unwanted bacterial formations, such as biofilm on their surfaces, which disrupt production lines or whole batches of e.g. biopharmaceuticals.

Accordingly, development of new methods for surface functionalization that prevent the formation of e.g. microbial communities on medical and other surfaces clearly have significant commercial potentials and socioeconomic benefits.

Along these lines, various methods have already been developed. Thus, WO 2010/075590 discloses medical implants comprising surfaces modified with a PEG polymer said polymer having a bioactive agent covalently attached via an amide bond. It is described that the bioactive agent may elute into the host body. No specific release mechanism is described, but the agent will elute slowly under physiological conditions [0056].

Likewise, Gomes, J. et al., Chem. Comm., 2013, 49, 155 discloses a titanium surface modified with a bioactive agent (a Quorum Sensing modulator) via a molecular anchor and a linker. Again the bioactive agent is attached via amide bonds and the agent will elute from the surface slowly under physiological conditions. No specific release mechanism is described.

In Xiong, M.-H. et al., J. Am. Chem. Soc., 134(9), 4355 a nanoparticle is disclosed which comprises a bioactive agent in the center of the micellar nanoparticle which is partially based on poly(ε-caprolactone) (PCL). The PCL is disrupted upon contact with bacterial lipases leading to release of the bioactive agent. It is not clear whether the nanoparticles are e.g. cytotoxic before or after enzymatic disruption of the particle.

US 2009/227980 (Kangas, Steve et al.) discloses methods of releasing drugs, such as antimicrobial agents, biofilm inhibitors, antibiotics, etc. from a surface [0038] of a balloon catheter. A method is suggested where drug is attached to the surface of the balloon via a hydrolysable ester bond, and an esterase is suggested as a possible trigger for the cleavage of the ester. The method is generically described and is not demonstrated by any examples and the use of esters with adjacent fatty acid alkyl derivatives are not suggested.

US 2010/098738 (Milner, Richard et al.) discloses implantable metal devices having an active biosurface comprising surface bound bioactive agents via reactive functional groups. It is suggested that the bioactive agent may have an enzyme labile bond to the polymer [0111]. Esters or esters with adjacent fatty acid alkyl derivatives are not suggested as enzyme labile bonds.

US2008/207535 (Urban, Marek et al.) discloses chemically modified polymers, with acid groups on the polymer surface. A PEG linker is attached to the modified polymers via amidation or esterification and antibiotics are linked to the PEG linker [e.g. FIG. 1 and FIG. 9]. Nothing is disclosed regarding enzymatic release of the bioactive compounds, and it is clear that the compounds stay attached to the surface [e.g. FIG. 2]. Esters with adjacent fatty acid alkyl derivatives are not disclosed as enzyme labile bonds.

Hence, improved devices and methods which prevents the growth of cell populations, such as e.g. bacterial biofilms, on surfaces would be advantageous, and in particular devices and methods for inhibiting cell growth on surfaces where the bioactive inhibitor agents are very efficiently and selectively released ‘on demand’ via enzymatic cleavage, would be advantageous.

### SUMMARY OF THE INVENTION

Thus, an object of the present invention relates to providing a polymer, or a surface modified with a polymer, that is covalently attached to a bioactive agent which is released upon enzymatic cleavage.

In particular, it is an object of the present invention to provide a surface as explained above wherein the bioactive agent is attached to the polymer via an ester bond which is sensitive to cleavage by an enzyme that solves the above mentioned problems of the prior art with less controlled release under physiological conditions and release via nanoparticles which must be adhered to a surface by further means, and which may potentially be harmful or toxic before or after disruption of the nanoparticle structure.

Thus, one aspect of the invention relates to a medical device comprising a device substrate having a surface, a polymer coating attached to said surface, and a bioactive agent covalently attached to said polymer coating, and

wherein said bioactive agent covalently attached to said polymer coating is selected from the substrates of formulas (I)-(II)

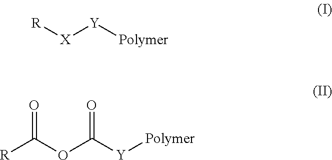

wherein
X is selected from

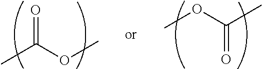

R(COOH) or ROH is the bioactive agent,
Y is —(CH₂)ₙ— wherein n is an integer between 2-25, or a fatty acid alkyl derived from a natural fatty acid.

Another aspect of the present invention relates to a method of inhibiting or preventing cell growth on a surface comprising modifying said surface with a polymer coating comprising a bioactive agent covalently attached to said polymer coating,

wherein said bioactive agent covalently attached to said polymer coating is selected from the substrates of formulas (I)-(II)

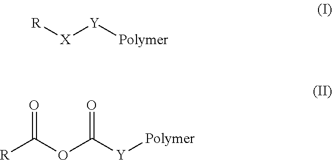

wherein
X is selected from

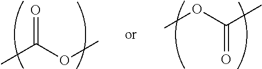

R(COOH) or ROH is the bioactive agent,
Y is —(CH₂)ₙ— wherein n is an integer between 2-25, or a fatty acid alkyl derived from a natural fatty acid.

The present inventors have surprisingly found that devices and methods as described above provides an “on demand” release of bioactive agents, such as for example antibiotics, by providing these bioactive agents covalently attached to a surface via ester or carboxylic acid anhydride bonds that are cleaved in the presence of certain enzymes released by e.g. a host or a bacterium. This effectively leads to the eradication of bacteria in the presence of such a surface. It was surprisingly found that a fatty acid type alkyl chain adjacent to the ester or carboxylic acid anhydride provides for very effective and selective release in the presence of bacteria.

### BRIEF DESCRIPTION OF THE FIGURES

FIG. 1 shows an exemplary model of the enzyme triggered release of bioactive agents.

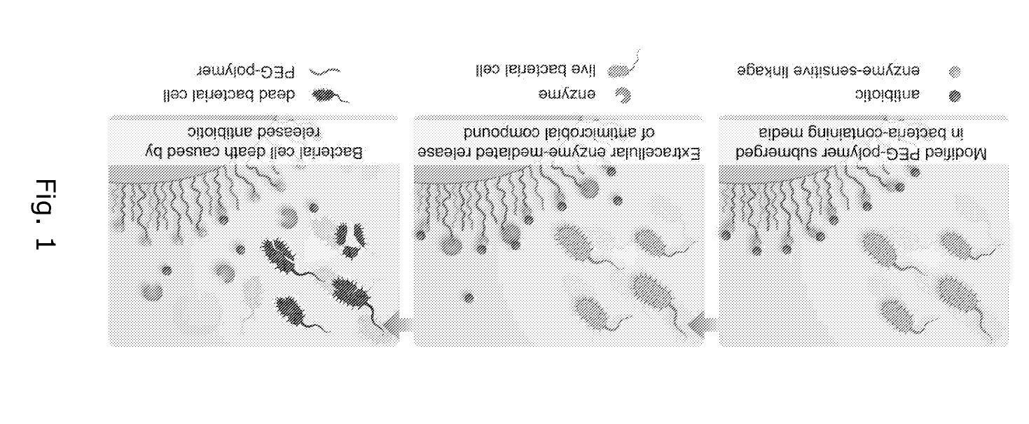

FIG. 2 shows the structure of the ChemMatrix resin used as the polymer base.

FIG. 3 shows the solid-phase synthesis of AHL-release precursor. i) Fmoc(OTrt)homoserine (3 equiv.), 1-(Mesitylene-2-sulfonyl)-3-nitro-1,2,4-triazole (MSNT) (2.25 equiv.), N-methylimidazole (3 equiv.), DMF, 1 h, rt; ii) 20% piperidine (DMF), 5 and 30 min, rt; iii) butanoyl chloride (5 equiv.), NEt₃ (10 equiv.), CH₂Cl₂, 1 h, rt; iv) 5% TFA (CH₂Cl₂), 1 h, rt; v) octanoyl chloride (5 equiv.), triethylamine (10 equiv.), DMAP (0.15 eq.), CH₂Cl₂, 1 h, rt.

FIG. 4 shows Boc-protection of ciprofloxacin 7 via treatment with Boc₂O and NaOH in dioxine to provide protected ciprofloxaxin 8 (FIG. 4A). The synthesis of azelaic anhydride 10 from azelaic acid 9 is shown below (FIG. 4B). The solid-phase synthesis of antibiotic-release precursor 13 was achieved as depicted in FIG. 4C), where the conditions were: i) HMBA-ChemMatrix resin was treated with 10 (3 equiv.), N-methyl imidazole (3 equiv.), CH₂Cl₂, 2 h to obtain substrate 11; ii) Substrate 11 was treated with 8 (3 equiv.), bis(trichloromethyl)carbonate (1 equiv.), triethylamine (5 equiv.), CH₂Cl₂, 2 h to obtain substrate 12; iii) Substrate 12 was treated with TMSOTf (5 equiv.), CH₂Cl₂, 2 h to remove the Boc protecting group and obtain substrate 13, i.e. a polymer bound bioactive agent.

FIG. 5 shows the enzymatic or base mediated cleavage of ciprofloxacin and azelaic acid from a polymer support (resin 13).

FIG. 6 shows RP UPLC-MS chromatograms of clevage products from resin 13 (base treatment). Peak 5—ciprofloxacin (ESI-MS calculated for C₁₇H₁₈FN₃O₃, 331.3. found M+H 332.3); Peak 6—azelaic acid (ESI MS calculated for C₉H₁₆O₄ 188.1. found M−1 187.2).

FIG. 7 shows RP UPLC-MS chromatograms of cleavage products from resin 13 (lipase treatment). Peak 6—ciprofloxacin (ESI-MS calculated for C₁₇H₁₈FN₃O₃, 331.3. found M+H 332.3); Peak 8—azelaic acid (ESI MS calculated for C₉H₁₆O₄ 188.1. found M−1 187.2).

FIG. 8 shows bacteria-triggered release of AHL 6. Step vi) is the lipase-mediated ester hydrolysis while step vii) is the cyclative release of AHL 6.

FIG. 9 shows the BHL-dependant activation of the ahyR/ahyI-gfp reporter system in E. coli. The graph demonstrates that BHL is released from Beads 4 in the presence of the beads and lipase, whereas it is not released from beads alone or lipase alone or from a blank experiment (growth medium alone).

FIG. 10 shows the viability of P. aeruginosa wild-type strain and lipA lipC estA triple mutant in the presence 0.00 μg of Beads 13 (i.e. no beads) over 4 hours.

FIG. 11 shows the viability of P. aeruginosa wild-type strain and lipA lipC estA triple mutant in the presence 0.03 μg of Beads 13 over 4 hours.

FIG. 12 shows the viability of P. aeruginosa wild-type strain and lipA lipC estA triple mutant in the presence 0.06 μg of Beads 13 over 4 hours.

FIG. 13 shows the viability of P. aeruginosa wild-type strain and lipA lipC estA triple mutant in the presence 0.09 μg of Beads 13 over 4 hours.

FIG. 14 shows resin 14 comprising amide bonded ciprofloxacin, with an ester bond present between the polymer and a very short alkyl linker —CH₂—.

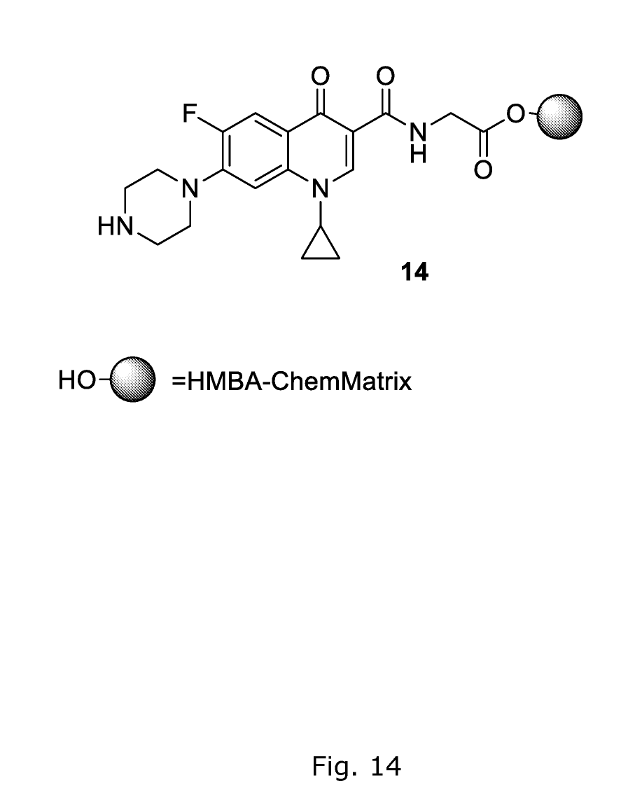

FIG. 15 shows the viability of wild-type P. aeruginosa in the presence of growth media, beads 14, beads 14+lipase, chemMatrix resin and chemMatrix+lipase. It is shown that beads 14 have no antibacterial effect, even in the presence of added lipase, which is also the case for the polymer resin alone.

The present invention will now be described in more detail in the following.

### DETAILED DESCRIPTION OF THE INVENTION

### Definitions

Prior to discussing the present invention in further details, the following terms and conventions will first be defined:

### Medical Device

In the present context a “medical device” in the broadest sense is any device used in the medical industry. This includes devices that are put in direct contact with human or animal bodies but also devices or device parts that are otherwise used in e.g. a hospital setting. Medical devices include macroscopic devices, such as e.g. catheters or stents and also microscopic devices such as e.g. various beads, including polymeric beads.

### Device Substrate

In the present context “device substrate” refers to the underlying substrate forming the base or surface on which the polymer coating of the present invention is attached. The device substrate may be formed by any material capable of forming a base for a polymer coating. Examples of such substrates include but are not limited to metals, alloys, polymers, plastics, concrete, glass, carbon, rubbers and natural substrates including but not limited to graphite, bone, wood, rock, or cellulose.

### Polymer Coating

In the present context “polymer coating” is defined as a layer of polymeric material fully or partially covering a substrate layer underneath. The polymer coating may be attached to the substrate by any possible means. If the device substrate is a polymer, the polymer coating may simply be the top layer of the substrate polymer. Polymers may include functional groups used to covalently bind to linker or bioactive agents.

### Bioactive Agent

In the present context a bioactive agent in the broadest sense is any agent capable of interaction with a biological species. Examples of such agents include but are not limited to medicinal products, bio-fluorescent compounds, toxins, antimicrobials, antibiotics and disinfectants.

### Attached and Covalently Attached

In the present context “attached” refers to any kind of attachment between two substrates such as e.g. a polymer to a surface. This may include but is not limited to attachment via electrostatic forces, hydrogen bonding, ionic bonding, Wan der Waals forces, hydrophobic or hydrophilic interactions, or covalent bonding. In the present context “covalently attached” refers to attachment via covalent chemical bonds.

### Ester and Carboxylic Acid Anhydride Moiety

In the present context a “moiety” is a chemical functional group. Thus, in the present context an “ester moiety” is represented by the formula C—[C(O)O]—C, where the terminal carbons may be substituted in any way, while an “carboxylic acid anhydride moiety” refers to the functional group C—[C(O)OC(O)]—C where the terminal carbons may be substituted in any way. It is to be understood that the bond sensitive to cleavage by an enzyme is the ester or anhydride bond, i.e. the bond between a carbonyl carbon (C(O) above) and the non-carbonyl oxygen. In particular cases the ester may be a phosphoric acid ester.

### Enzyme

In the present context “enzyme” is any catalytic entity capable of catalysing chemical reactions, particularly an enzyme capable of cleaving an ester or a carboxylic acid anhydride bond. Enzymes may be artificial or natural and are typically in the form of a protein or peptide.

In an effort to meet the increasing need for inhibiting undesirable cell growth on the surfaces of e.g. medical devices and other surfaces the present inventors have investigated the possibility of attaching bioactive agents covalently to polymers which are often used to coat the substrate surfaces of such devices. The inventors have surprisingly found that when applying particular ester or carboxylic acid anhydride moieties for the attachment of such bioactive agents, these may be released into the surrounding environment “on demand” as they are cleaved in the presence of enzymes, which may for example be released by the unwanted cells or by other means, including physiological responses.

Thus, a first aspect of the present invention is a medical device comprising a device substrate having a surface, a polymer coating attached to said surface, and a bioactive agent covalently attached to said polymer coating, and

wherein said bioactive agent is covalently attached to said polymer coating via at least one ester or carboxylic acid anhydride moiety sensitive to cleavage by an enzyme.

An alternative aspect of the present invention is a medical device comprising a device substrate having a surface, a polymer coating attached to said surface, and a bioactive agent covalently attached to said polymer coating, and

wherein said bioactive agent is covalently attached to said polymer coating via at least one ester or carboxylic acid anhydride moiety.

I a preferred embodiment the above medical device is defined with the proviso that if the device substrate is a polymer the polymer coating may be the outer polymer layer of said device substrate.

The ester or carboxylic acid anhydride moiety sensitive to cleavage by an enzyme may preferably be in a position which means that the cleavage of the ester or anhydride results in the direct liberation of the active bioactive agent. This means that the bioactive agent preferably should comprise a carboxylic acid, phosphoric acid or a hydroxyl group, since these are the two groups formed upon e.g. hydrolytic enzymatic cleavage of an ester or anhydride bond. Thus in a preferred embodiment the bioactive agent comprises a carboxylic acid, phosphoric acid or a hydroxyl group. Preferably this carboxylic acid, phosphoric acid or hydroxyl group is therefore used as the basis of the ester or anhydride formed when attaching said bioactive agent to a polymer coating via a linkage according to the present invention. Even more preferably the bioactive agent comprises a carboxylic acid or a hydroxyl group, most preferably a carboxylic acid.

The undesirable cell growth on surfaces such as on a medical device may often be microbial cell growth, i.e. caused by for example bacteria, algae or fungi. Antimicrobials will inhibit the growth of such cells upon release from a surface. Thus, the bioactive agent may preferably be an antimicrobial. Bacteria are a particular concern and thus in a preferred embodiment said antimicrobial is an antibiotic. In an even more preferred embodiment said antimicrobial is an antibiotic comprising a carboxylic acid, phosphoric acid or a hydroxyl group.

The antibiotic comprising a carboxylic acid, phosphoric acid or a hydroxyl group may thus preferably be selected from the group consisting of Platensimycin, Fusidic acid, Loracarbef, Ertapenem, Doripenem monohydrate, Imipenem, Daptomycin, Aztreonam, Vancomycin, Cefadroxil, Cefazolin, Cefalotin, Cefalexin, Cefaclor, Cefamandole, Cefoxitin, Cefprozil, Cefuroxime, Cefixime, Cefditoren, Cefoperazone, Cefotaxime, Ceftazidime, Ceftibuten, Ceftizoxime, Ceftriaxone, Cefepime, Amoxicillin, Ampicillin, Azlocillin, Carbenicillin, Ciprofloxacin, Cloxacillin, Dicloxacillin, Flucloxacillin, Mezlocillin, Meticillin, Oxacillin, Nafcillin, Benzylpenicillin, Phenoxymethylpenicillin, Piperacillin, Temocillin, Ticarcillin, Enoxacin, Gatifloxacin, Levofloxacin, Lomefloxacin, Moxifloxacin, Nalidixic acid, Norfloxacin, Ofloxacin, Sulfasalazine, Ceftaroline fosamil, Ceftobiprole, Telavancin, Tobramycin, Kanamycin, Teicoplanin, Torezolid, Ethambutol, and Metronidazole.

In a preferred embodiment the polymer coating is made from a polymer suitable for use on the surface of a medical device. Such a polymer may preferably be a polymer selected from the group consisting of Polyethylene glycol, polyethylene, polyethylene terephthalate, polystyrene, polypropylene, poly(methyl methacrylate), polysulfone, polyphosphazene, polydimethoxysiloxane, polyacrylamide, polyether etherketone, polyetherimide, polyvinyl chloride, and polylactic acid. The polymer coating may preferably be a brush type polymer coating, wherein the individual polymer chains are attached to the substrate surface at one end. In a preferred embodiment at least 0.1% of the individual polymer chains of the polymer coating are covalently attached to a bioactive molecule, such as at least 0.5%, 1%, 2%, 5%, 10%, 20%, 50%, 70%, 80%, 90%, 95%, such as at least 99%. Polymer coatings for medical devices are well known to skilled person, and are described for example in Knetsch et al., Polymers, 2011, 3, 340-366. Preferably the polymer used has reactive groups which may be used for creating the linkage between the polymer and the linker-ester-(bioactive agent).

As mentioned the bioactive agent preferably comprises a carboxylic acid or hydroxyl group and this group preferably forms part of the ester or carboxylic anhydride moiety which is sensitive to enzyme cleavage. The ester or anhydride moiety may be linked to the polymer forming the polymer coating of the present invention via a bond or any suitable linker moiety. Thus, in a preferred embodiment said bioactive agent covalently attached to said polymer coating via at least one ester or carboxylic acid anhydride moiety sensitive to cleavage by an enzyme is selected from the substrates of formulas (I)-(II)

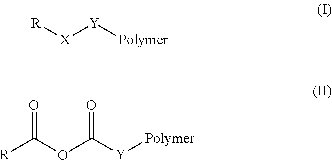

wherein
X is selected from

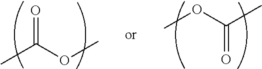

R(COOH) or ROH is the bioactive agent,
Y is a bond, or a linker moiety.

In a preferred embodiment the linker moiety is selected from the group consisting of an optionally substituted alkyl, an optionally substituted mono- or polyunsaturated alkyl, a polyethylene glycol, a fatty acid alkyl from a natural source, a polypeptide, a polysaccharide or any combination thereof. Optionally substituted alkyls may preferably have a chain length of 2 carbons or more. Optionally substituted alkyls may preferably be substituted by alkyl or hydroxyl.

The linker moiety may be attached to polymer by any suitable covalent bond. For example if the polymers terminal end comprises a primary amine, an amide bond may be a suitable link between the linker moiety and the polymer. Thus, in a preferred embodiment the linker moiety is attached to the polymer via an ester, amide, thioamide, amine, ether, thioether or triazole bond. Particularly preferred are esters and amide bonds. If an ester is used the linker moiety may be released in conjunction with the bioactive agent. The linker may in this case be a bioactive agent itself or it may be inactive. If release of the linker moiety is undesirable the linker moiety may preferably be attached to the polymer by an amide, amine, ether, thioether, or triazole bond.

Particularly useful linker moieties may include un-branched alkyl chains or alkyl chains derived from natural fatty acids. Thus, in a preferred embodiment Y is —(CH₂)ₙ—, wherein n is an integer between 0 and 30, such as 1-30, 2-25, 2-20, 2-15, 2-10, 3-10, such as 4-7 or a fatty acid alkyl derived from a natural fatty acid. The fatty acid alkyl derived from a natural fatty acid may preferable have a chain length of 2 carbons or more. When Y is —(CH₂)ₙ— it is to be understood that the polymer of formula (I) and (II) includes an attaching moiety as described above, including e.g. an ester, amide, thioamide, amine, ether, thioether or triazole.

In a particularly preferred embodiment said bioactive agent covalently attached to said polymer coating via at least one ester or carboxylic acid anhydride moiety sensitive to cleavage by an enzyme is selected from the substrates of formulas (III-IV)

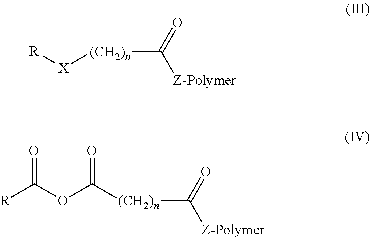

wherein
X is selected from

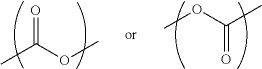

R(COOH) or ROH is the bioactive agent,
Z is selected from the group consisting of O, N, S, and CH₂,
n is an integer between 1 and 30.

Preferably Z is selected from the group consisting of O, N, and S. Even more preferably Z is O, and n is 2-25, 2-20, 2-15, 2-10, 3-10, such as 4-7. Yet even more preferably X is

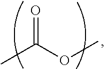

R(COOH) is the bioactive agent, Z is O, and n is 4-7.

The present inventors have surprisingly found that that esters with adjacent alkyl chains with n higher than 1 provide for more effective “on demand” release by enzymatic cleavage provided by extracellular bacterial enzymes, without increasing potentially undesirable release when bacteria are not present.

The present invention is envisioned to be particularly useful for the inhibition or prevention of bacterial growth on surfaces, including the surfaces of polymeric or polymer coated medical devices. Therefore, “on-demand” release of bioactive agents, particularly antibiotics may be achieved by providing an ester or carboxylic anhydride moiety for attachment which is sensitive to cleavage by enzymes produced by bacteria, particularly extracellular bacterial enzymes.

The enzyme cleaving the bioactive agent from the polymer surface may stem from the unwanted cells, e.g. bacterial cells, which may be present on or near the surface, but it may also for example be a host enzyme, e.g. released in response to the presence of bacterial cells. Also, the enzyme may be an enzyme added to the surface to release the bioactive agent. Thus, in a preferred embodiment the enzyme is an extracellular bacterial or host enzyme, preferably a bacterial enzyme. The enzyme may preferably be a lipase or esterase, most preferably a lipase.

Certain bacteria are capable of proliferating in a biofilm form, where the bacteria are present in an extracellular matrix. Intercellular communication, also designated quorum sensing, makes this form of bacterial growth particular resistant to e.g. antibiotics once the biofilm is formed. The inhibition of biofilm formation is therefore of particular interest. Thus in a preferred embodiment said enzyme is an enzyme produced by a bacterium capable of forming biofilm. In an even more preferred embodiment said enzyme is produced by bacteria in the biofilm form. Bacteria capable of forming biofilm include but are not limited to P. aeruginosa, E. coli, Klebsiella pneumoniae, S. aureus, and S. epidermidis. The bacterium may preferably be a multi-resistant strain of said bacterium.

The present invention is particularly relevant for medical devices which are brought into contact with living tissue, including e.g. the human body. Thus in a preferred embodiment said medical device is selected from the group consisting of implants, artificial organs, stents, surgical instruments, heart valves, and catheters.

The modified polymer surface of the present invention is generally applicable for the on-demand inhibition of cell growth on surfaces. There are many surfaces in both medical and industrial settings where unwanted cell growth occurs. Apart from medical devices, such surfaces may for example include the inner surfaces of tubes, pipelines, tank and reactors used in industry.

Thus, another aspect of the present invention is a method of inhibiting or preventing cell growth on a surface comprising modifying said surface with a polymer coating comprising a bioactive agent covalently attached to said polymer coating, wherein said bioactive agent is covalently attached to said polymer coating via at least one ester or carboxylic acid anhydride moiety sensitive to cleavage by an enzyme.

An alternative aspect of the present invention is a method of inhibiting or preventing cell growth on a surface comprising modifying said surface with a polymer coating comprising a bioactive agent covalently attached to said polymer coating,

wherein said bioactive agent is covalently attached to said polymer coating via at least one ester or carboxylic acid anhydride moiety.

Bacterial cells may be particularly undesirable on various surfaces and thus said cell growth may preferably be bacterial cell growth. Bacterial biofilm formation may be especially difficult to inhibit using traditional means and thus, the bacterial cell growth may preferably be in the form of bacterial biofilm.

In a preferred embodiment said surface is the surface of a means for the transportation or storage of liquids. Particularly, said means for the transportation or storage of liquids may preferably be a tube, pipeline, reactor, bioreactor or tank. Said liquid may preferably be an aqueous composition or an oil. Said surface may alternatively be the surface of a medical device.

The preferred embodiments pertaining to the medical device aspect of the present invention naturally also apply to the present method aspect. The method of the present invention is however not limited to medical devices but may be applied to any surface where cell growth is not desirable.

All patent and non-patent references cited in the present application, are hereby incorporated by reference in their entirety.

The invention will now be described in further details in the following non-limiting examples.

### EXAMPLES

### General Material and Methods

Solid-phase synthesis was carried out using plastic-syringe techniques using amino-functionalized ChemMatrix resin (loading app. 0.3 mmol/g) as a support and 4-hydroxymethylbenzoic acid (HMBA) as a linker. Analytical HPLC was conducted on a Water Alliance 2695 RP-HPLC system using a Symmetry® C-18 column (d 2.5 μm, 4.6×75 mm, column temp: 25° C. flow rate 1 mL/min) with detection at 215 nm and 254 nm. Eluents A (0.1% TFA in H₂O) and B (0.1% TFA in MeCN) were used in a linear gradient (100% A to 100% B) in a run time of 13 min. Analytical LC/MS (ESI) analysis was performed on a Waters AQUITY RP-UPLC system equipped with a diode array detector using an AQUITY UPLC BEH C-18 column (d 1.7 μm, 2.1×50 mm; column temp: 65° C.; flow: 0.6 mL/min). Eluents A (0.1% HCO₂H in H₂O) and B (0.1% HCO₂H in acetonitrile) were used in a linear gradient. The LC system was coupled to a SQD mass spectrometer. All solvents were of HPLC grade, and all commercially available reagents were used without further purification.

### General Procedure for Solid Phase Synthesis

### (1) Attachment of HMBA Linker to Amino-Functionalized ChemMatrix Beads

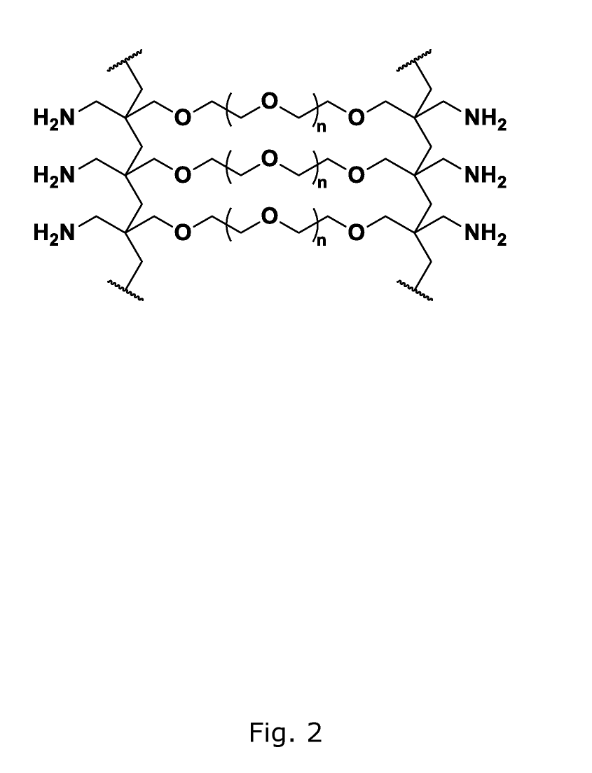

Attachment of the 4-hydroxymethylbenzoic acid (HMBA) linker to the amino-functionalized ChemMatrix resin (FIG. 2) resin was carried out by premixing HMBA (3 equiv.), NEM (4 equiv.), and TBTU (2.88 equiv.) for 5 min in DMF. The resulting solution was added to the resin and allowed to react for 2 h, followed by washing with DMF (×6), and CH₂Cl₂ (×6).

### (2) General Procedure for MSNT-Mediated Coupling of Fmoc-Protected Amino Acids to HMBA-Functionalized ChemMatrix Beads

Coupling of the first amino acid to the HMBA derivatized resin was accomplished by treating the freshly lyophilized resin with a mixture of the corresponding Fmoc-protected amino acid (3 equiv.), MeIm (5 equiv.), and MSNT (3 equiv.) in CH₂Cl₂. The support was washed with CH₂Cl₂ (×6) and the MSNT-mediated coupling procedure was repeated. The support was washed with CH₂Cl₂ and DMF (×6). Removal of the Fmoc protecting group was accomplished with 20% piperidine in DMF for 5 min. After washing twice with DMF, the deprotection procedure was repeated, now with a reaction time of 30 min. The resin was washed with DMF (×6), MeOH (×6) and CH₂Cl₂ (×6).

### Example 1

### Solid-Phase Synthesis of Quorum-Sensing Inducer Release System

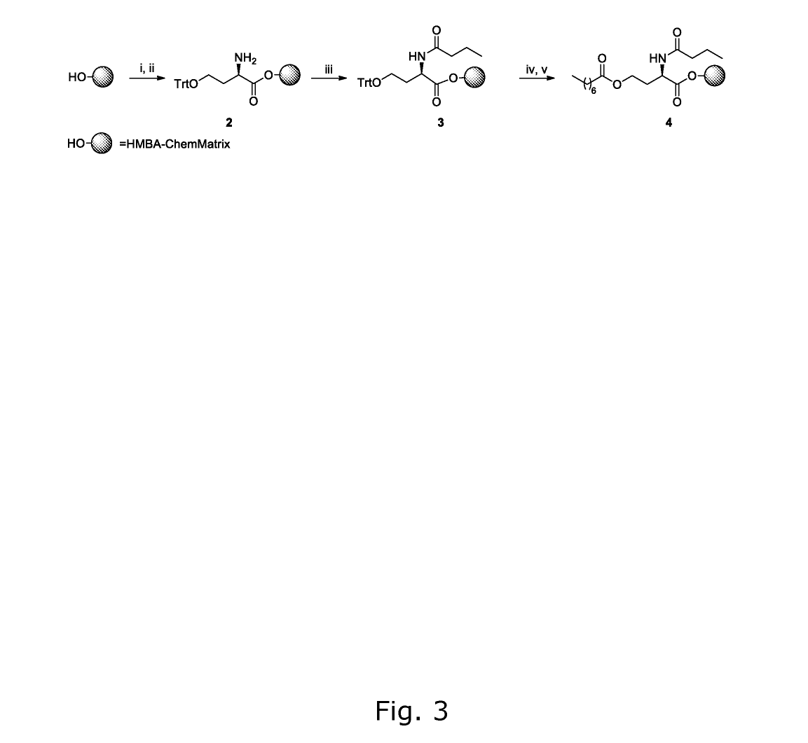

Fmoc(OTrt)homoserine was coupled onto the HMBA functionalized ChemMatrix resin according to the general solid-phase synthesis procedure (2). As depicted in FIG. 3, resin 2 (200 mg) was swelled in CH₂Cl₂ and butanoyl chloride (32 mL, 0.3 mmol, 5 equiv.) and NEt₃ (210 mL, 0.6 mmol, 10 equiv.) were added and allowed to react for 2 h. The resin was washed with DMF (×6), MeOH (×6) and CH₂Cl₂ (×6) and lyophilized. The resulting resin was swelled in 5% TFA-CH₂Cl₂ solution for 1 h. Beads were then filtered, washed with CH₂Cl₂ (×6), dried on air for 30 min and swelled again in CH₂Cl₂ and octanoyl chloride (52 mL, 0.3 mmol, 5 equiv.), DMAP (1 mg, 8 mmol, 0.15 equiv.) and NEt₃ (84 mL, 0.6 mmol, 10 equiv.) were immediately added. Resin was gently stirred and allowed to react for 1 h. The resin was washed with DMF (×6), MeOH (×6) and CH₂Cl₂ (×6) and lyophilized affording beads 4 which were then directly used in quorum sensing inducing experiments.

### Example 2

### Solid-Phase Synthesis of Polymer Bound Bioactive Agent

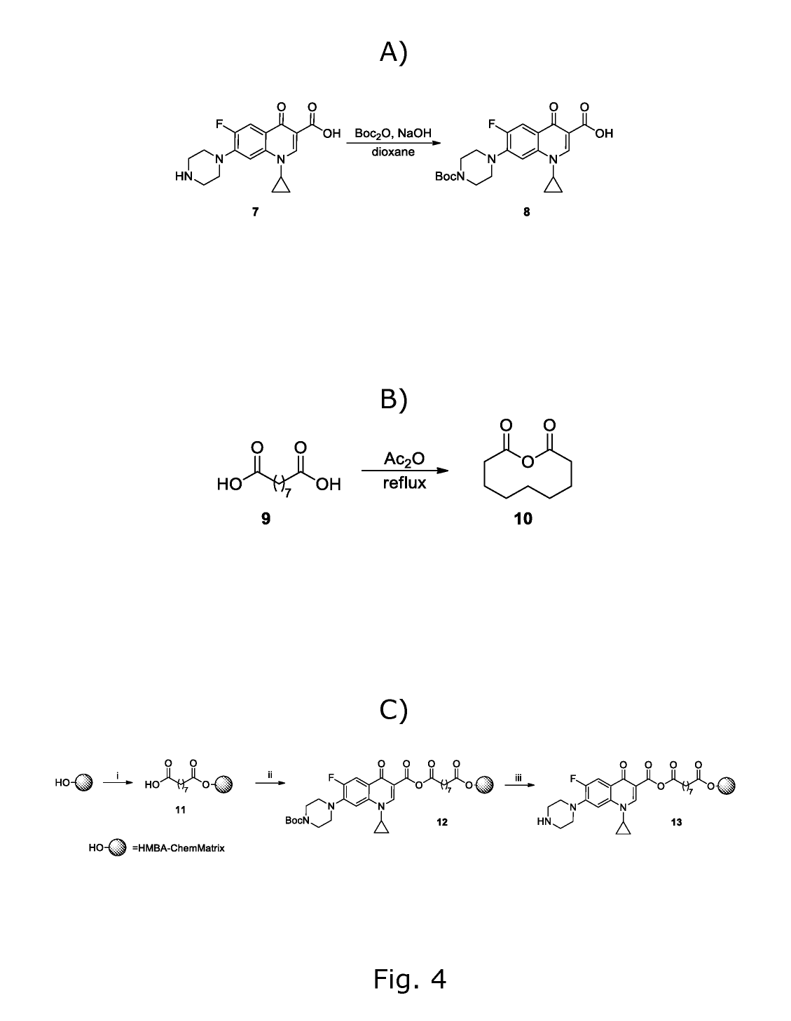

Ciprofloxacin 7 was Boc-protected to provide compound 8 according to the literature procedure provided in Tanaka et al. Bioorganic & medicinal chemistry, 2008, 16, 9217-29 (see FIG. 4A)).

Azelaic acid 9 (10 g, 0.05 mol) was refluxed in acetic anhydride for 6 h. Acetic anhydride was then removed in vacuo, residue was co-evaporated with toluene and vacuum dried overnight. Obtained product 10 was used without any further purification (see FIG. 4B)). The product was isolated as white solid, mp 55-57° C. ¹H NMR (300 MHz, CDCl₃): δ ppm 1.28 (m, 6H), 1.58 (m, 4H), 2.38 (t, J=7.4 Hz, 4H). IR (ATR): 1806 cm⁻¹ and 1741 cm⁻¹ (anhydride bands).

The solid-phase synthesis of antibiotic-release precursor 13 was achieved as depicted in FIG. 4C), where the conditions were: HMBA-linked ChemMatrix resin (200 mg) was swelled in CH₂Cl₂ and azelaic anhydride 10 (33.8 mg, 0.18 mmol, 3 equiv.) was added together with N-methyl imidazole (23 μL, 0.18 mmol. 3 equiv.). Resin was gently stirred an left at the room temperature. After 2 with DMF (×6), MeOH (×6) and CH₂Cl₂ (×6) and lyophilized affording resin 11. Boc-protected ciprofloxacin 8 (77.5 mg, 0.18 mmol, 1 equiv.) was dissolved in CH₂Cl₂ (1 ml) and BTC (25.8 mg, 0.06 mmol, 1 equiv.) and triethylamine (42 μL, 0.3 mmol, 5 equiv.) was added consequently. After 5 min mixture was added to the resin 11 and allowed to react for 2 hrs, then was washed with DMF (×6), MeOH (×6) and CH₂Cl₂ (×6) and lyophilized. Resulting resin was swelled in CH₂Cl₂ and TMSOTf (54 μL, 0.3 mmol, 5 equiv.) was added. Resin was allowed to react for 2 h, washed with DMF (×6), MeOH (×6) and CH₂Cl₂ (×6) and lyophilized affording beads 13, i.e. a polymer bound bioactive agent (see FIG. 4C)).

### Example 3

### Enzyme and Base Promoted Release of Bioactive Agent

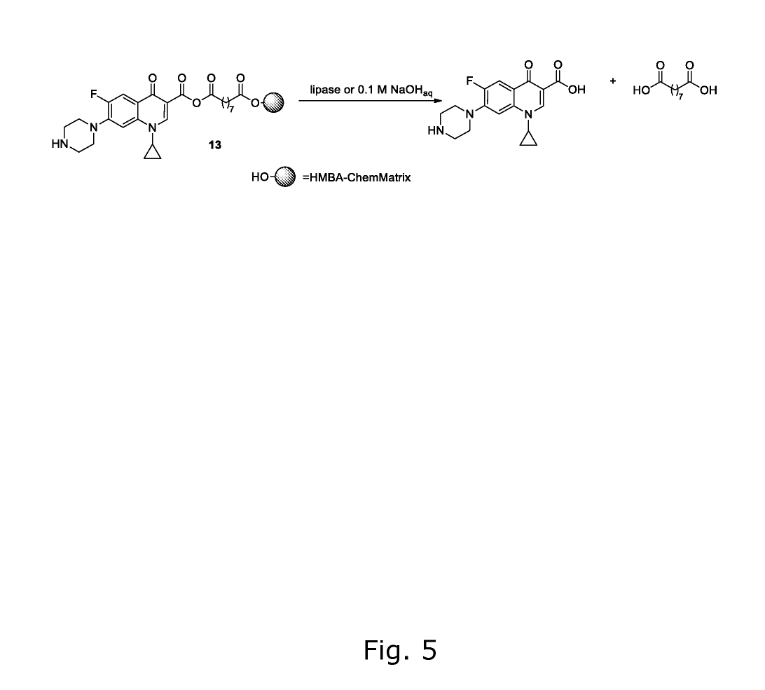

The Enzyme and base promoted release of bioactive agent is demonstrated via the lipase and NaOH promoted release of ciprofloxacin and azelaic acid from resin 13 as depicted in FIG. 5.

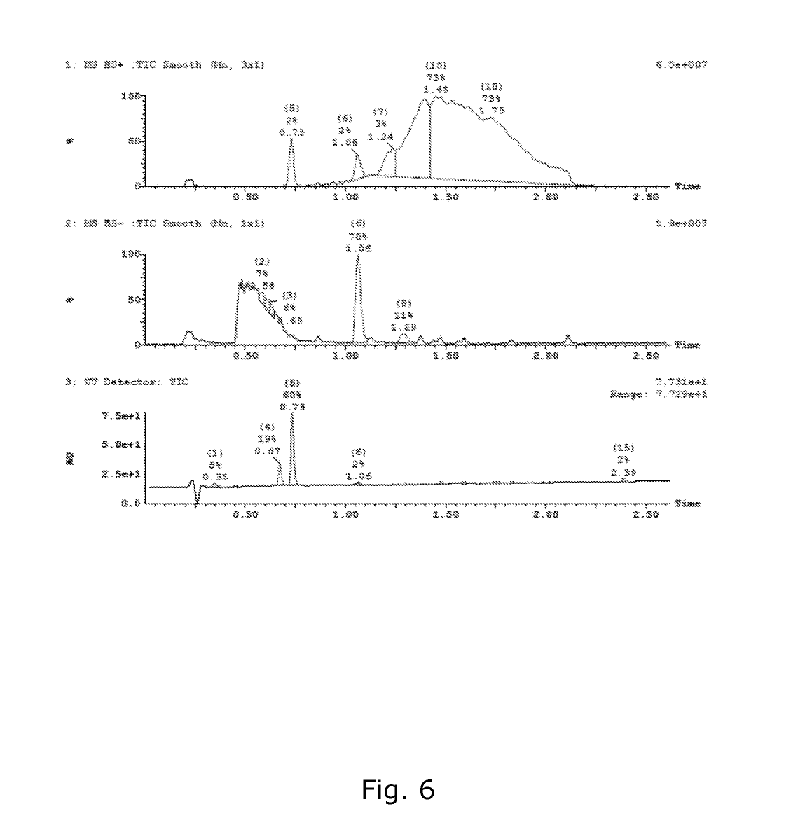

Thus FIG. 6 shows RP UPLC-MS chromatograms of cleavage products from resin 13 using base treatment. Herein Peak 5 is ciprofloxacin (ESI-MS calculated for C₁₇H₁₈FN₃O₃, 331.3. found M+H 332.3); and Peak 6 is azelaic acid (ESI MS calculated for C₉H₁₆O₄ 188.1. found M−1 187.2). Under neutral conditions resin 13 was found to be stable, i.e. no ciprofloxacin or azelaic acid was released.

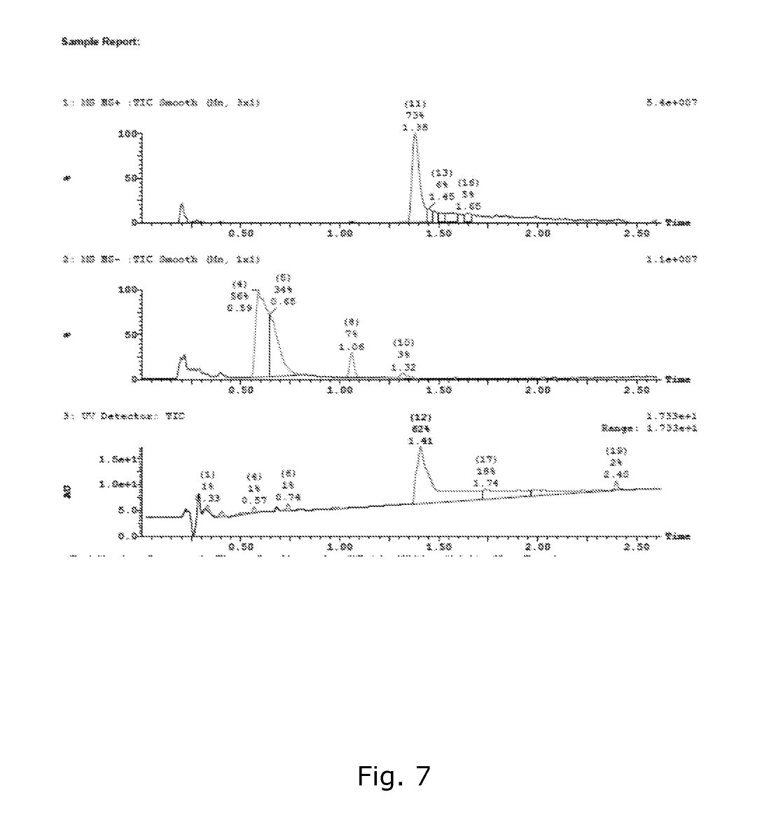

Similarly FIG. 7 shows RP UPLC-MS chromatograms of cleavage products from resin 13 using lipase treatment. Herein Peak 6 is ciprofloxacin (ESI-MS calculated for C₁₇H₁₈FN₃O₃, 331.3. found M+H 332.3); and Peak 8 is azelaic acid (ESI MS calculated for C₉H₁₆O₄ 188.1. found M−1 187.2).

The experiment demonstrates that the enzyme sensitive ester bonds of resin 13 will cleave in the presence of a lipase to afford the same products as those produced by base induced ester cleavage.

### Example 4

### Test of Quorum Sensing Induction of Beads 4

Given the importance of quorum sensing and cell-cell signaling for the biofilm infection process, we decided to use lactone 6 as a structural basis for a release system that, when triggered under carefully controlled conditions, would be expected to affect Gram-negative bacteria, such as Pseudomonas aeruginosa, using N-acyl L-homoserine lactone (AHL) inducers for activation of their QS systems.

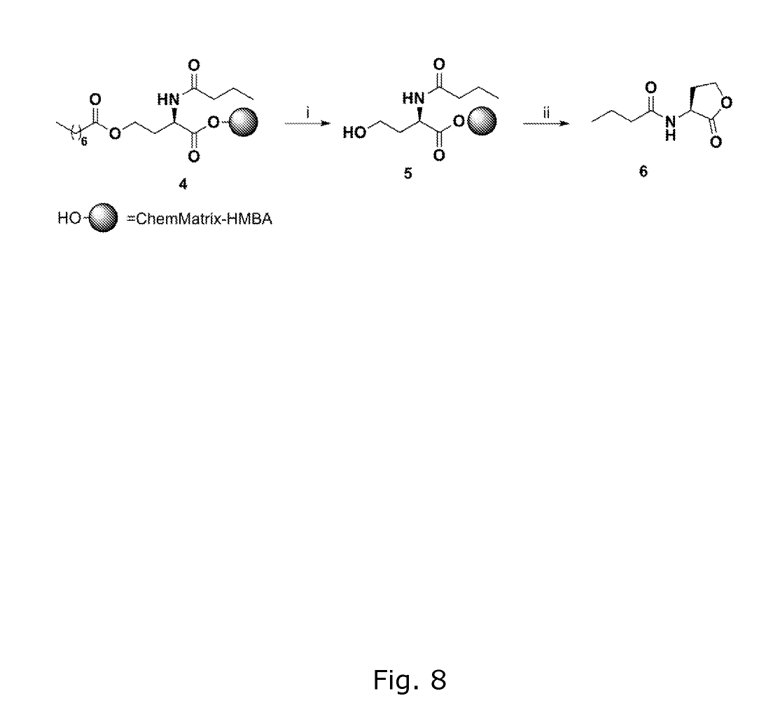

The homoserine-containing construct 4 was assembled on HMBA-linked ChemMatrix resin, which serves as a representative model system for PEG-based materials. Like related materials, this polymeric system also allows diffusion of biological macromolecules, such as enzymes and substrates. We envisioned how lipase-treatment of 4 upon ester hydrolysis would liberate a free hydroxyl group in resin 5 and undergo a spontaneous cyclization, possibly further catalysis by the acidic interior of the lipase, and form lactone 6 (see FIG. 8).

To test the concept we employed the E. coli MH205 as a QS monitor strain. This monitor contains an ahyR/ahyI-gfp reporter system, which responds readily to the presence of extracellular N-butanoyl-L-homoserine lactone 6 (BHL). The E. coli strain itself provides a lipase negative background.

We prepared a set of experiments where beads 4 were suspended in bacterial growth medium in concentrations corresponding roughly to 100 μM active liberated BHL (resin loading is app. 0.4 mmol/g).

Cultures in wells of polystyrene microtitre trays (Thermo Fisher Scientific, USA) (330 μL per well) were started by diluting Escherichia coli MH205 overnight cultures to OD₄₅₀=0.05 in AB medium supplemented with 100 μg/mL ampicillin, 0.5% glucose and 0.5% casamino acids, and were exposed to the following chemicals where indicated: 8.8 μM BHL (Sigma-Aldrich, Germany), 0.9 μg/μL lipase from Pseudomonas fluorescens (Sigma-Aldrich, Germany), and beads 4 (0.1 μg/μl). The cultures were incubated at 37° C., and OD₄₅₀ and Gfp fluorescence was measured continuously by the use of a Wallac microplate reader (Perkin Elmer, USA). As references, a 2.5 μM solution of BHL supplemented growth medium as well as an un-supplemented growth medium were included as controls.

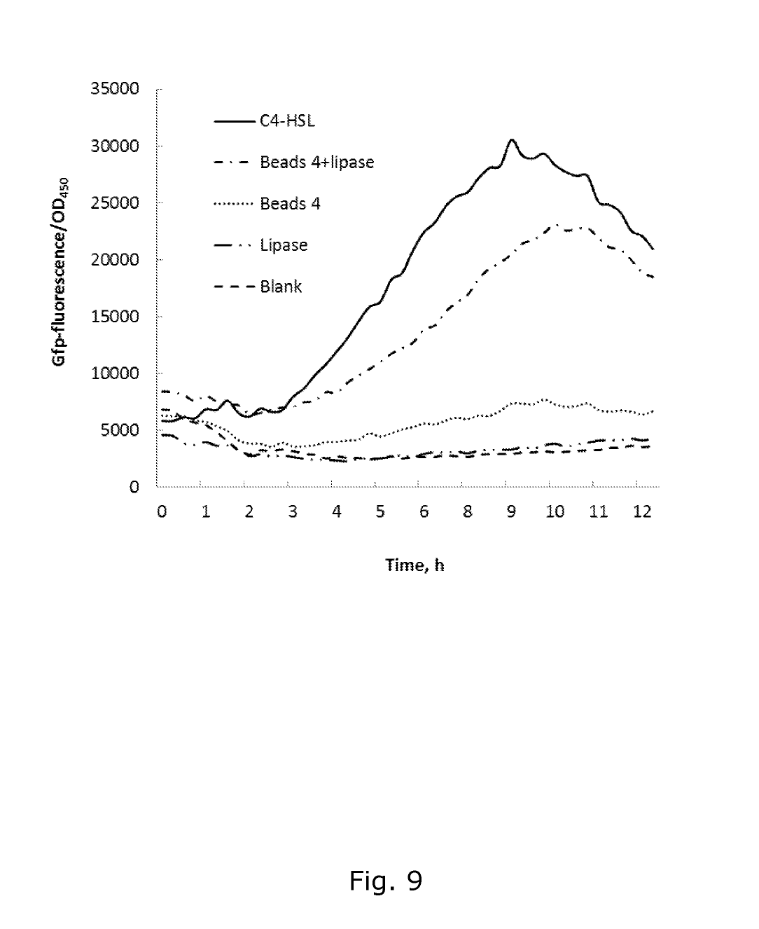

Rewardingly, beads 4 and the presence of lipase was indeed found to be necessary for ample activation of the genetically engineered QS monitor MH205, compared with medium devoid of added lipase and the un-supplemented media (FIG. 9).

### Example 5

### Test of Antibacterial Activity of Beads 13

We assumed that the presence of an extracellular bacterial lipase would catalyse the hydrolysis of the mixed anhydride bond and thus liberate the antibiotics. Lipase is known to be produced at infectious sites. E.g. antisera obtained from CF patients with increasing duration of P. aeruginosa infection contained increasing amounts of anti-lipase, indicating the presence of P. aeruginosa lipase in the infected patient.

As to this end, we investigated if the antibiotic-coated beads 13 can kill a lipase-producing biofilm-forming bacterium. The opportunistic pathogen Pseudomonas aeruginosa produces and secretes the two lipases LipA and LipC, and the outer membrane-located esterase EstA. We assessed the viability (colony forming units/ml) of P. aeruginosa bacteria in cultures supplemented with beads 13. As a negative control we used a P. aeruginosa lipA lipC estA triple mutant which is unable to produce the extracellular LipA, LipC, and EstA lipolytic enzymes.

The Pseudomonas aeruginosa wild type and lipase defective mutant lipAlipCestA were grown in LB medium at 37° C. For the test of antibacterial activity, P. aeruginosa LB overnight cultures were diluted to OD₆₀₀=0.1 in fresh LB medium, and transferred to microtiter trays (Thermo Fisher Scientific, USA) (330 μl pr well). Cultures in wells were exposed to beads 13 with the concentrations of 0, 0.03, 0.06, or 0.09 μg/μl. The trays were incubated at 37° C. for 4 hours. For the determination of colony forming units (CFU/ml) in the multiwell cultures, vigorously vortexed serial dilutions of cell suspensions were plated on LB agar plates every 1 hour, and colonies were counted after overnight incubation at 37° C.

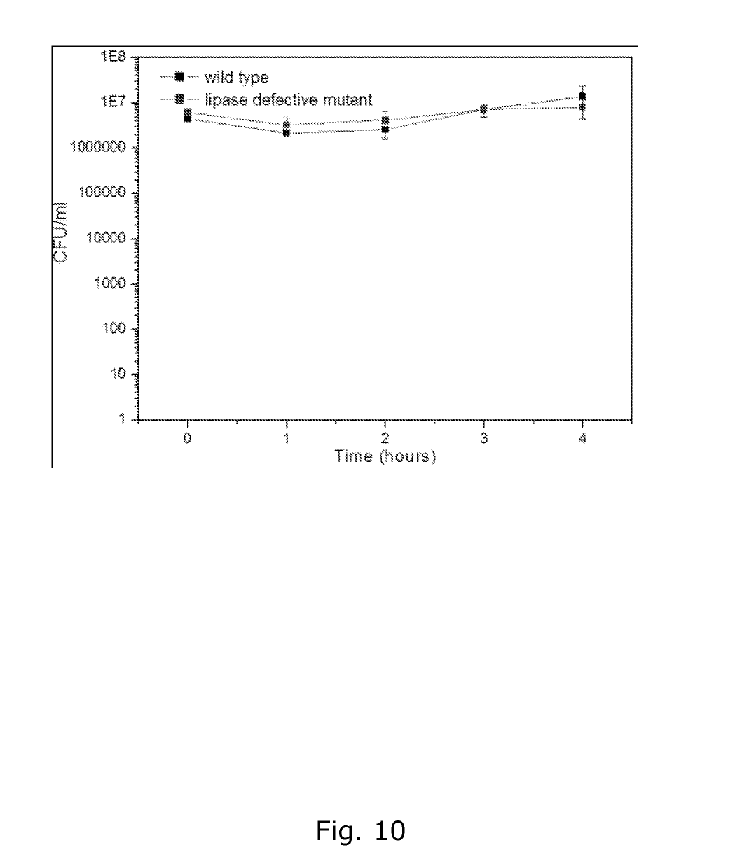

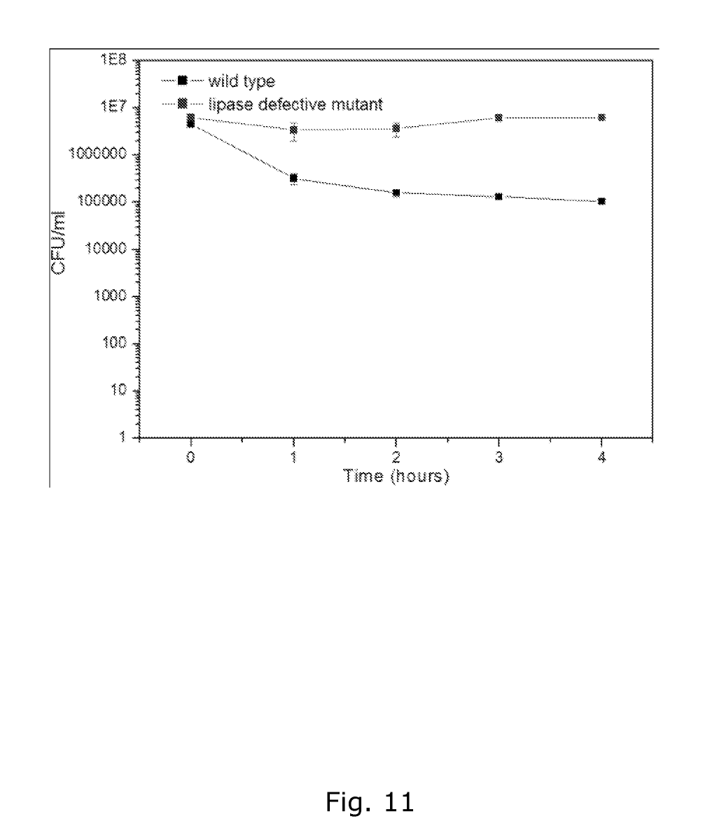

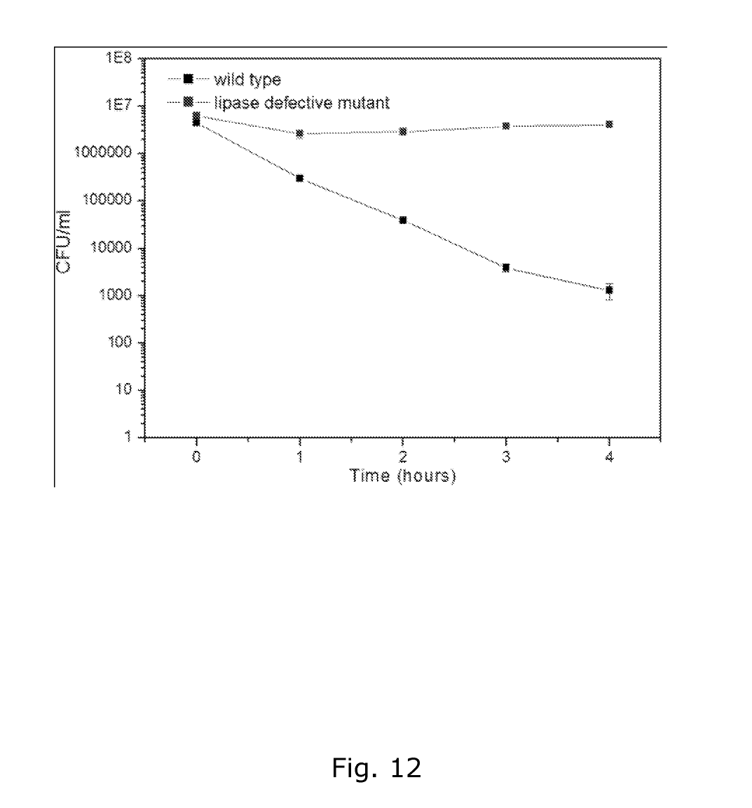

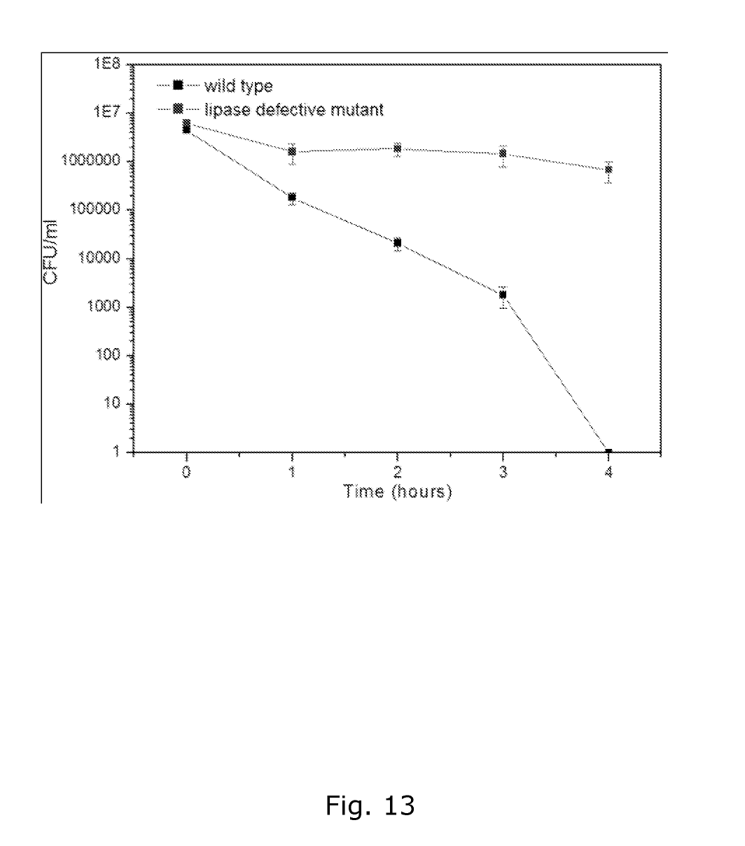

Thus FIGS. 10-13 shows the Survival (CFU/ml) of the Pseudomonas aeruginosa wild type and lipase defective mutant, lipAlipCestA, in the presence of different amounts of beads 13, i.e. FIG. 10: no beads; FIG. 11: 0.03 μg beads/μl; FIG. 12: 0.06 μg beads/pi; and FIG. 13: 0.09 μg beads/μl.

Thus the data of FIGS. 10-13 demonstrate that the wild-type strain completely killed itself in the presence 0.09 μg beads/μl of beads 13 within 4 hours, whereas the population of the lipase-defective mutant was only insignificantly decreased in the presence of beads 13.

### Example 6

### Test of Antibacterial Activity of Beads 14 and the Importance of Adjacent Alkyl Chains to the Enzyme Cleavable Moiety

Amide bonded ciprofloxacin beads 14 were synthesized as follows.

HMBA derivatized ChemMatrix resin (200 mg) was swelled in CH₂Cl₂ and treated with a mixture of the FmocGlyOH (53 mg, 0.18 mmol, 3 equiv.), MeIm (25 μL, 0.3 mmol, 5 equiv.), and MSNT (53 mg, 0.18 mmol, 3 equiv.) in CH₂Cl₂ (500 μL). The support was washed with CH₂Cl₂ (×6) and the MSNT mediated coupling procedure was repeated. The support was washed with CH₂Cl₂ and DMF (×6). Removal of the Fmoc protecting group was accomplished with 20% piperidine solution in DMF for 5 min. After washing twice with DMF, the deprotection procedure was repeated, now with a reaction time of 30 min. The resin was washed with DMF (×6), MeOH (×6) and CH₂Cl₂ (×6), and lyophilized.

Boc-protected ciprofloxacin (77.5 mg, 0.18 mmol, 3 equiv.), NEM (30 μL, 0.24 mmol, 4 equiv.), and TBTU (55 mg, 0.17 mmol, 2.88 equiv.) were premixed for 10 min in DMF (500 μL) and resulting solution was added to the glycine-HMBA modified ChemMatrix resin (200 mg) and allowed to react for 2 h. The resin was washed with DMF (×6), MeOH (×6) and CH₂Cl₂ (×6) and lyophilized. The resulting resin was swelled in CH₂Cl₂ and TMSOTf (54 μL, 0.3 mmol, 5 equiv.) was added. Resin was allowed to react for 2 h, washed with DMF (×6), MeOH (×6) and CH₂Cl₂ (×6) and lyophilized affording beads 14. Pseudomonas aeruginosa wild type LB overnight cultures were diluted to OD₆₀₀=0.1 in fresh LB medium, and transferred to microtiter trays (Thermo Fisher Scientific, USA) (330 μl pr well). Cultures in wells were exposed to beads 14 with the concentration 0.09 μg/μl. The trays were incubated at 37° C., and for the determination of colony forming units (CFU/ml) in the multiwell cultures, vigorously vortexed serial dilutions of cell suspensions were plated on LB agar plates after incubation times 0 h, 3 h, and 20 h, and colonies were counted after overnight incubation at 37° C.

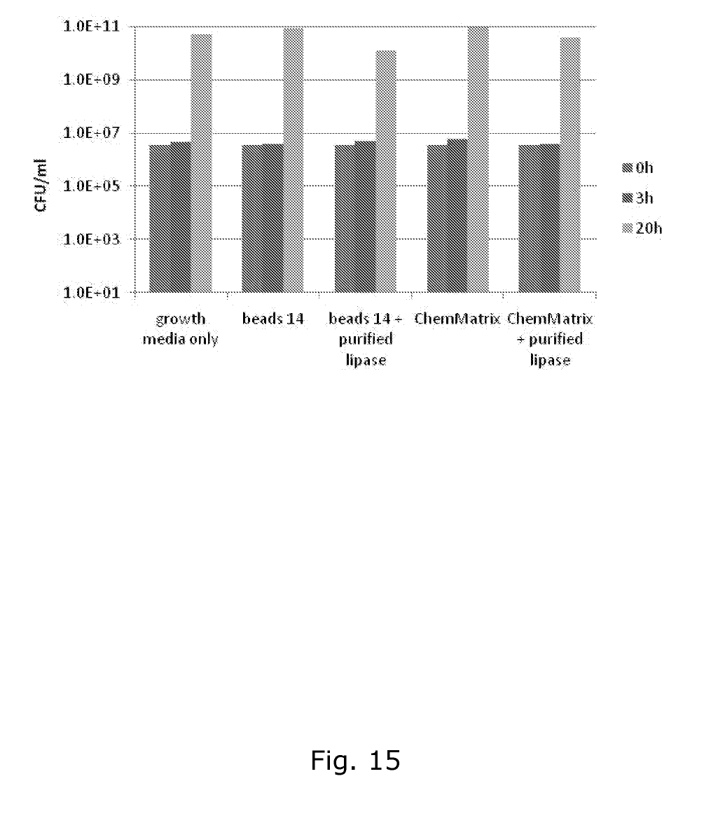

Thus, FIG. 15 shows the viability (CFU/ml) of the P. aeruginosa wild-type in the presence of ChemMatrix resin and ChemMatrix modified with amide-bonded ciprofloxacin 14. This data demonstrates that the ChemMatrix resin itself, and ChemMatrix modified with amide-bonded ciprofloxacin (i.e. where the drug cannot be cleaved by the lipase or the growth media) did not show any antibiotic effect, neither against the wild-type strain, nor for the lipase-defective mutant.

It should be noted, however, that although the ciprofloxacin in 14 is amide bonded, an ester bond does in fact exist between the antibiotic and the polymer bead. The complete lack of antibiotic activity of 14 in the presence of wild-type bacteria indicates that ester bonds without adjacent alkyl chains of a length under 2 carbons are difficult to cleave for these enzymes. This makes alkyl esters and corresponding alkyl acid anhydrides a highly selective linker for enzyme induced release of antibiotics from surfaces.

### REFERENCES

WO 2010/075590
Gomes, J. et al., Chem. Comm., 2013, 49, 155
Xiong, M.-H. et al., J. Am. Chem. Soc., 134(9), 4355
Knetsch et al., Polymers, 2011, 3, 340-366.
Tanaka et al. Bioorganic & medicinal chemistry, 2008, 16, 9217-29

1. A medical device comprising a device substrate having a surface, a polymer coating attached to said surface, and a bioactive agent covalently attached to said polymer coating, and
wherein said bioactive agent covalently attached to said polymer coating is selected from the substrates of formulas (I)-(II) 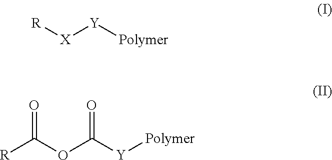
wherein
X is selected from 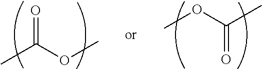
R(COOH) or ROH is the bioactive agent,
Y is —(CH₂)ₙ— wherein n is an integer between 2-25, or a fatty acid alkyl derived from a natural fatty acid.

2-49. (canceled)

50. A medical device according to claim 1, wherein said bioactive agent is an antimicrobial.

51. A medical device according to claim 50, wherein said antimicrobial is an antibiotic.

52. A medical device according to claim 51, wherein said antimicrobial is an antibiotic comprising a carboxylic acid, phosphoric acid or a hydroxyl group.

53. A medical device according to claim 51, wherein said antibiotic is selected from the group consisting of Platensimycin, Fusidic acid, Loracarbef, Ertapenem, Doripenem monohydrate, Imipenem, Daptomycin, Aztreonam, Vancomycin, Cefadroxil, Cefazolin, Cefalotin, Cefalexin, Cefaclor, Cefamandole, Cefoxitin, Cefprozil, Cefuroxime, Cefixime, Cefditoren, Cefoperazone, Cefotaxime, Ceftazidime, Ceftibuten, Ceftizoxime, Ceftriaxone, Cefepime, Amoxicillin, Ampicillin, Azlocillin, Carbenicillin, Ciprofloxacin, Cloxacillin, Dicloxacillin, Flucloxacillin, Mezlocillin, Meticillin, Oxacillin, Nafcillin, Benzylpenicillin, Phenoxymethylpenicillin, Piperacillin, Temocillin, Ticarcillin, Enoxacin, Gatifloxacin, Levofloxacin, Lomefloxacin, Moxifloxacin, Nalidixic acid, Norfloxacin, Ofloxacin, Sulfasalazine, Ceftaroline fosamil, Ceftobiprole, Telavancin, Tobramycin, Kanamycin, Teicoplanin, Torezolid, Ethambutol, and Metronidazole.

54. A medical device according to claim 1, wherein said polymer coating is made from a polymer selected from the group consisting of Polyethylene glycol, polyethylene, polyethylene terephthalate, polystyrene, polypropelene, poly(methyl methacrylate), polysulfone, polyphosphazene, polydimethoxysiloxane, polyacrylamide, polyether etherketone, polyetherimide, polyvinyl chloride, and polylactic acid.

55. A medical device according to claim 1, wherein Y is attached to the polymer via an ester, amide, thioamide, amine, ether, thioether or triazole bond.

56. A medical device according to claim 1, wherein Y is (CH₂)ₙ—, wherein n is an integer between 2-20, 2-15, 2-10, 3-10, such as 4-7 or a fatty acid alkyl derived from a natural fatty acid.

57. A medical device according to claim 1, wherein said bioactive agent covalently attached to said polymer coating is selected from the substrates of formulas (III-IV) 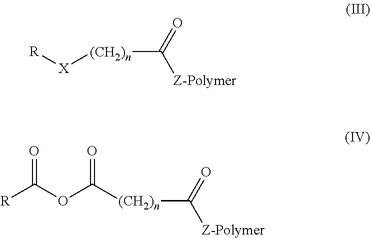
wherein
X is selected from 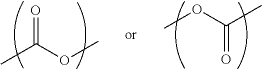
R(COOH) or ROH is the bioactive agent,
Z is selected from the group consisting of O, N, S, and CH₂,
n is an integer between 2 and 30.

58. A medical device according to claim 57, wherein Z is selected from the group consisting of O, N, and S.

59. A medical device according to claim 57, wherein, Z is O, and n is 2-25, 2-20, 2-15, 2-10, 3-10, such as 4-7.

60. A medical device according to claim 57, wherein, X is 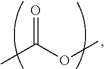
R(COOH) is the bioactive agent, Z is O, and n is 4-7.

61. A medical device according to claim 1, wherein the enzyme is an extracellular bacterial or host enzyme.

62. A medical device according to claim 61, wherein the enzyme is an extracellular bacterial enzyme.

63. A medical device according to claim 62, wherein said enzyme is an enzyme produced by a bacterium capable of forming biofilm.

64. A medical device according to claim 63, wherein said enzyme is an enzyme produced by bacterium in biofilm form.

65. A medical device according to claim 63, wherein said biofilm forming bacterium is selected from the group consisting of P. aeruginosa, E. coli, Klebsiella pneumoniae, S. aureus, and S. epidermidis.

66. A medical device according to claim 63, wherein said biofilm forming bacterium is a multi-resistant strain of said bacterium.

67. A medical device according to claim 1, wherein the enzyme is a lipase or esterase, preferably a lipase.

68. A medical device according to claim 1, wherein said polymer coating is a brush type polymer coating, wherein individual polymer chains are attached to the substrate surface at one end.

69. A medical device according to claim 68, wherein at least 0.1% of the individual polymer chains of the polymer coating are covalently attached to a bioactive molecule, such as at least 0.5%, 1%, 2%, 5%, 10%, 20%, 50%, 70%, 80%, 90%, 95%, such as at least 99%.

70. A medical device according to claim 1, wherein said medical device is selected from the group consisting of implants, artificial organs, stents, surgical instruments, heart valves, and catheters.

71. A method of inhibiting or preventing cell growth on a surface comprising modifying said surface with a polymer coating comprising a bioactive agent covalently attached to said polymer coating,
wherein said bioactive agent covalently attached to said polymer coating is selected from the substrates of formulas (I)-(II) 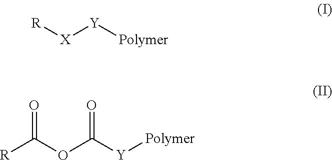
wherein
X is selected from 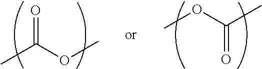
R(COOH) or ROH is the bioactive agent,
Y is —(CH₂)ₙ— wherein n is an integer between 2-25, or a fatty acid alkyl derived from a natural fatty acid.

72. A method according to claim 71, wherein said cell growth is bacterial cell growth.

73. A method according to claim 72, wherein said bacterial cell growth is in the form of bacterial biofilm.

74. A method according to claim 71, wherein said surface is the surface of a means for the transportation or storage of liquids.

75. A method according to claim 74, wherein said means for the transportation or storage of liquids is a tube, pipeline, reactor, bioreactor or tank.

76. A method according to claim 74, wherein said liquid is an aqueous composition or an oil.

77. A method according to claim 71, wherein said surface is the surface of a medical device.

78. A method according to claim 71, wherein said bioactive agent is an antimicrobial.

79. A method according to claim 78, wherein said antimicrobial is an antibiotic.

80. A method according to claim 79, wherein said antimicrobial is an antibiotic comprising a carboxylic acid, phosphoric acid or a hydroxyl group.

81. A method according to claim 80, wherein said antibiotic is selected from the group consisting of Platensimycin, Fusidic acid, Loracarbef, Ertapenem, Doripenem monohydrate, Imipenem, Daptomycin, Aztreonam, Vancomycin, Cefadroxil, Cefazolin, Cefalotin, Cefalexin, Cefaclor, Cefamandole, Cefoxitin, Cefprozil, Cefuroxime, Cefixime, Cefditoren, Cefoperazone, Cefotaxime, Ceftazidime, Ceftibuten, Ceftizoxime, Ceftriaxone, Cefepime, Amoxicillin, Ampicillin, Azlocillin, Carbenicillin, Ciprofloxacin, Cloxacillin, Dicloxacillin, Flucloxacillin, Mezlocillin, Meticillin, Oxacillin, Nafcillin, Benzylpenicillin, Phenoxymethylpenicillin, Piperacillin, Temocillin, Ticarcillin, Enoxacin, Gatifloxacin, Levofloxacin, Lomefloxacin, Moxifloxacin, Nalidixic acid, Norfloxacin, Ofloxacin, Sulfasalazine, Ceftaroline fosamil, Ceftobiprole, Telavancin, Tobramycin, Kanamycin, Teicoplanin, Torezolid, Ethambutol, and Metronidazole.

82. A method according to claim 71, wherein said polymer coating is made from a polymer selected from the group consisting of polyethylene glycol, polyethylene, polyethylene terephthalate, polystyrene, polypropelene, poly(methyl methacrylate), polysulfone, polyphosphazene, polydimethoxysiloxane, polyacrylamide, polyether etherketone, polyetherimide, polyvinyl chloride, and polylactic acid.

83. A method according to claim 71, wherein the linker moiety is attached to the polymer via an ester, amide, thioamide, amine, ether, thioether or triazole bond.

84. A method according to claim 71, wherein Y is —(CH₂)ₙ—, wherein n is an integer between 2-25, 2-20, 2-15, 2-10, 3-10, such as 4-7 or a fatty acid alkyl derived from a natural fatty acid.

85. A method according to claim 71, wherein said bioactive agent covalently attached to said polymer coating is selected from the substrates of formulas (III-IV) 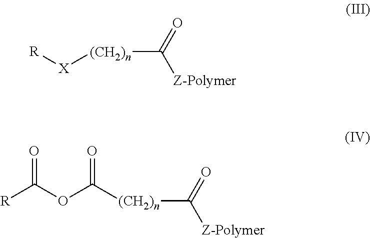
wherein
X is selected from 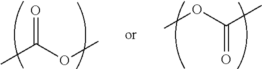
R(COOH) or ROH is the bioactive agent,
Z is selected from the group consisting of O, N, S, and CH₂,
n is an integer between 2 and 30.

86. A method according to claim 85, wherein Z is selected from the group consisting of O, N, and S.

87. A method according to claim 86, wherein, Z is O, and n is 2-25, 2-20, 2-15, 2-10, 3-10, such as 4-7.

88. A method according to claim 87, wherein, X is 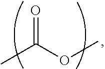
R(COOH) is the bioactive agent, Z is O, and n is 4-7.

89. A method according to claim 71, wherein the enzyme is an extracellular bacterial or host enzyme.

90. A method according to claim 89, wherein the enzyme is an extracellular bacterial enzyme.

91. A method according to claim 90, wherein said enzyme is an enzyme produced by a bacterium capable of forming biofilm.

92. A method according to claim 91, wherein said enzyme is an enzyme produced by bacterium in biofilm form.

93. A method according to claim 91, wherein said biofilm forming bacterium is selected from the group consisting of P. aeruginosa, E. coli, Klebsiella pneumoniae, S. aureus, and S. epidermidis.

94. A method according to claim 91, wherein said biofilm forming bacterium is a multi-resistant strain of said bacterium.

95. A method according to claim 71, wherein the enzyme is a lipase or esterase, preferably a lipase.

96. A method according to claim 71, wherein said polymer coating is a brush type polymer coating, wherein individual polymer chains are attached to the substrate surface at one end.

97. A method according to claim 96, wherein at least 0.1% of the individual polymer chains of the polymer coating are covalently attached to a bioactive molecule, such as at least 0.5%, 1%, 2%, 5%, 10%, 20%, 50%, 70%, 80%, 90%, 95%, such as at least 99%.
